(19) United States
(12) Patent Application Publication
(10) Pub. No.: US 2005/0003526 A1
(43) Pub. Date: Jan. 6, 2005

(54) Pig mast cell cultures and uses thereof

(76) Inventors: Francoise Femenia, Saint-Maur-des-Fosses (FR); Claude Delouis, Jouy-en-Josas (FR); Andre Laurent Parodi, Saint-Maur-la-Varenne (FR); Thomas Lilin, Maisons Alfort (FR); Michel Arock, Paris (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/492,950

(22) PCT Filed: Oct. 22, 2002
(86) PCT No.: PCT/FR02/03616

(30) Foreign Application Priority Data
Oct. 22, 2001 (FR) 01 13608

(51) Int. Cl. G01N 33/53; C12N 5/06; G01N 33/567

(52) U.S. Cl. 435/325; 435/7.2

(57) ABSTRACT

The invention concerns a method for obtaining pure pig mast cell cultures by culturing stem cells derived from the liver and the fetal marrow in the presence of IL-3 and SCF. The invention also concerns pig mast cell lines derived from said cultures.

The invention relates to pig mast cells cultures and to uses thereof.

Mast cells are cells of the immune system which are involved in the inflammatory response, in particular in allergic and hypersensitivity phenomena. They are located in the connective tissue, in particular in the skin and in the digestive and respiratory tracts, and in the intestinal and respiratory mucus membranes. A small number of mast cells also exists in the bone marrow and in the lymphoid organs.

Mature mast cells, whatever their location, have certain characteristics in common, such as the presence of numerous intracytoplasmic metachromatic granulations. They are rounded cells which are 13 to 22 μm in diameter, they have a single rounded nucleus which is central or, most commonly, off-center. The cytoplasm is completely filled with granulations, the abundance of granulations sometimes covering part of the nucleus.

These granulations contain various chemical substances synthesized by the mast cells, among which mention will in particular be made of histamine, serotonin, proteoglycanas such as heparin or chondroitin sulfate, enzymes, in particular proteases, cytokines, such as TNF-alpha, and “chemoattractant” factors, eosinophils and neutrophils (ABRAHAM and MALAVIYA, Infection and Immunity, 65, 3501, 1997). These pro-inflammatory substances are released abruptly (phenomenon referred to as “degranulation”) during mast cell activation. A secondary response is subsequently set up, associated with the de novo synthesis of mediators such as leukotrienes, prostaglandins and PAF (platelet activating factor), but also of interleukins (IL4, ILS, IL6, IL10, IL12, IL13), of cytokines (beta-TGF, gamma-IFN, GM-CSF) and of chemokines (MCP-1, IL8, RANTES) (BEFUS, Reg. Immunol., 2,176,1989; MOQBEL et al., Immunology, 60, 425, 1987). All these factors actively contribute to the triggering of an inflammatory process and to the setting up of a T-lymphocyte-dependent specific immune response.

Mast cells in fact constitute a very heterogeneous cell population.

Two distinct mast cell subpopulations which exhibit very different biochemical and functional characteristics have been characterized: mucosal mast cells and serosal mast cells. These two subpopulations can be differentiated by the active substances produced and stored in the granules. Thus, in mice, mucosal mast cells produce mainly histamine, chymase and chondroitin sulfate A and E, and serosal mast cells produce histamine, serotonin, chymase, tryptase and heparin.

In humans, mucosal mast cells, also referred to as MC_{T} (tryptase +) mainly produce histamine, tryptase, heparin and chondroitin sulfate A and E, and serosal mast cells, also referred to as MC_{TC} (tryptase +and chymase +), also produce chymase.

Mast cells are derived from hematopoietic precursors (GALLI, Lab. Invest. 62,5-33,1990). Mast cell populations have been obtained from mouse bone marrow cultures (RAZIN et al., J. Biol. Chem., 257,7229-7236, 1982; DAYTON et al., Proc. Natl. Acad. Sci. U.S.A., 85, 569-572,1988) in the presence of conditioned media or in the presence of IL3. A pure population of mast cells is obtained from a suspension of mouse hematopoietic cells in 3 weeks.

Obtaining human mast cells cultures in vitro has proved to be very difficult. Cultures of bone marrow cells or of umbilical cord cells in the presence of IL3 have led to the appearance of basophilic granulocytes (TADOKORO et al., J. Exp. Med., 158,857-871,1983). The co-culturing of human umbilical cord cells with mouse 3T3 fibroblasts has, however, made it possible to obtain mature mast cells after culturing for four weeks (ISHIZAKA et al., Current Opinion in Immunology, 5, 937-943,1993; FURITSU et al., Proc. Natl. Acad. Sci. U.S.A., 86,10039-10043,1989). It has subsequently been shown (ISHIZAKA et al., abovementioned publication) that the differentiation and the proliferation of human mast cells in vitro depends on the c-kit ligand (also referred to as SCF for: “Stem Cells Factor”) produced by the 3T3 cells.

Mast cell cultures constitute a useful tool for studying the mechanisms involved in various inflammatory and/or immune phenomena, for example in the context of the allergic response, or of the immune response to attack by various pathogenes, in particular parasites. However, to date, mast cell cultures have been successfully differentiated and maintained in vitro only in the case of human or rodent cells; in addition, only a small number of mast cell lines are currently available.

The aim of the present invention is to provide mast cell cultures originating from the pig, and mast cell lines derived from these cultures. In fact, besides its economic interest, the pig constitutes a model which is increasingly used in the context of physiological and clinical studies. Furthermore, pigs harbor parasites which are transmissible to humans (Trichinella, cysticercus, toxoplasm), and the study of the role played by mast cells in the mechanisms of defense against these parasites is of great value from the point of view of animal health and of public health (medical prophylaxis).

Previous studies have shown that the establishment of human and rodent mast cells lines only isolated from tumors (mastocytomes) requires the presence of a single specific cytokine in the culture medium.

The presence of pig mastocytomes is an extremely rare occurrence, which has to date prevented mast cell lines being obtained from this species.

The inventors have now succeeded in developing a method of culturing, in the presence of two specific cytokines, which makes it possible to obtain a pure population of porcine mast cells from hematopoietic precursors of the liver or of the bone marrow, and to maintain it in culture for a very long period of time.

They have also succeeded in obtaining, from this culture, immortalized cells which conserve the characteristics of the initial mast cells.

Consequently, a subject of the present invention is a method for obtaining a porcine mast cell culture, characterized in that it comprises placing in culture stem cells obtained from pig fetal liver or bone marrow, in a medium comprising at least 0.2 ng/ml, advantageously from 0.5 to 10 ng/ml, preferably from 1 to 5 ng/ml, and entirely preferably approximately 2 ng/ml, of porcine IL-3 and at least 8 ng/ml, advantageously from to 20 to 200 ng/ml, preferably from 40 to 120 ng/ml and entirely preferably approximately 80 ng/ml, of porcine SCF.

According to a preferred embodiment of the present invention, the cells are cultured in this medium for at least 60 to 70 days so as to obtain 100% of mast cells.

According to another preferred embodiment of the present invention, said method also comprises transforming said culture with an immortalizing oncogene.

A subject of the present invention is also any porcine mast cell culture which can be obtained using the method in accordance with the invention.

At least 90%, preferably at least 95%, and entirely preferably at least 99%, of the cells of this culture have the following characteristics:
they contain granulations containing histamine and heparin;
they express the c-kit receptor and the IgE receptor.

The present invention encompasses in particular any subpopulation, line or clone of mast cells which can be obtained from the porcine mast cell cultures in accordance with the invention.

The term “culture” here denotes, in general, a cell or a set of cells cultured in vitro. A culture developed directly from a cell or tissue sample taken from an animal is referred to as “primary culture”. The term “line” is used once at least one passage, and generally several consecutive passages in subculture, have been successfully performed, and denotes any culture which is derived therefrom. The term “clone” denotes a set of cells derived from a single cell of a primary culture or of a line (SCHAEFFER, In Vitro Cellular and Developmental Biology, 26,91-101,1990).

Subjects of the present invention are thus more particularly:
a line of mast cells derived from pig fetal liver stem cells, deposited with the CNCM (Collection Nationale de Cultures de Microorganismes [French National Collection of Cultures and Microorganisms], Institut Pasteur, 26 rue du Dbcteur Roux, 75724 PARIS CEDEX 15, France) on October 17,2001, under the number I-2735;
a line of mast cells derived from pig fetal liver stem cells and transfected with the SV40 virus T oncogene, deposited with the CNCM on October 17,2001, under the number I-2736;
a line of mast cells derived from pig fetal bone marrow stem cells and transfected with the SV40 virus T oncogene, deposited with the CNCM on October 17,2001, under the number I-2734.

All these cells can be cultured under the conditions defined above.

The maintaining in culture does not however require the presence of IL-3, and the SCF can be used as doses lower than those used to obtain the cultures. Thus, the SCF can be used at concentrations starting from 2 ng/ml, preferably at concentrations of 5 to 100 ng/ml, advantageously from 8 to 80 ng/ml. These cells can also be maintained in culture in the absence of SCF and of IL-3 for approximately 2 months.

The mast cells in accordance with the invention can in particular be used as models for studying mast cell activation in response to an allergen or to a parasite. They are most particularly advantageous as an in vitro model for studying the immune mechanisms which are involved in acquiring protective immunity with respect to parasitic nematodes. By way of example, mention will be made of the study of the mechanisms involved in the setting up of local immunity after infestation of the intestinal epithelial cells with Trichinella.

The mast cells in accordance with the invention can also be used for identifying the antigens involved in mast cell activation not only in pigs, but also in other animals, including humans, in particular in the context of the screening of antigens which can potentially be used to obtain vaccines or diagnostic reagents. Recombinant or synthetic antigens can thus in particular be tested on the basis of their ability to activate these mast cells in vitro.

In the case of Trichinella for example, these antigens may have applications for the diagnosis of trichinellosis in animals (early diagnosis of trichinellosis in the Suidae), or for the development of vaccines (for example mucosal vaccination).

The present invention will be understood more clearly from the further description which follows, which makes reference to nonlimiting examples describing the obtaining of porcine mast cell cultures and lines in accordance with the invention.

### EXAMPLE 1

### Isolation of Pure Populations of Mast Cells From Pig Fetal Marrow or Liver Sampling and Placing Cells in Culture

The samples were taken from 7 fetuses of a Meishan sow on the 77^{th} day of gestation; 7 livers and 8 femurs were taken.

Harvesting Liver Cells The livers were ground and the cells were individualized with 20 ml of 0.5% trypsin, 5 mM EDTA, in a waterbath at 37° C. for 15 min. After incubation, filtration through sterile gauze pads was carried out, performing several rinses with DMEM medium (GIBCO) supplemented with 15% FCS, 100 U/ml of penicillin/streptomycin Glutamin (complete DMEM medium).

After centrifugation at 1000 g for 5 min., a ficoll gradient was used in order to remove the red blood cells; the pellet is taken up in 25 ml of DMEM medium for 17 ml of ficoll. Centrifugation was carried out at 1500 g for 25 min., the ring of white cells is then taken and rinsed twice in complete DMEM medium.

The pellet was then recovered in 10 ml of MEMα medium (GIBCO) and a cell count was performed (i.e. 2×10⁶ cells/ml).

The cells were seeded, in a proportion of 4×10⁶ cells/ml, into a flask with 5 ml of MEMα medium supplemented with 15% FCS (fetal calf serum), 100 U/ml of penicillin/streptomycin, 2 mM glutamin, and PGE2 (prostaglandin E, Sigma) at a final concentration of 1 μM (complete MEMA medium), in the presence of recombinant porcine IL3 (2 ng/ml) and SCF (80 ng/ml) (BIOTRANSPLANT).

Harvesting Bone Marrow Cells

The fetal femurs were cut at each end with scissors and the bone marrow was harvested by perfusion using a syringe containing PBS.

Having been rinsed in PBS, the cells were seeded, in a proportion of 2.4×10⁶ cells/ml, into a flask with 20 ml of complete MEMα medium in the presence of porcine IL3 (2 ng/ml) and porcine SCF (80 ng/ml).

Cell Culture

The flasks are placed in an incubator at 37° C. in the presence of CO₂ (at 5%).

The cells are re-seeded every week, in a proportion of 10⁶ cells/ml, in complete MEMα medium in the presence of porcine IL3 (2 ng/ml) and porcine SCF (80 ng/ml).

Samples are taken at regular intervals in order to perform a cell count. After cytospinning and staining with toluidine blue, the percentage of mast cells is obtained by counting under a microscope.

The results are given in table I below.

TABLE I

| Age of the | Bone marrow | | Liver | |
|---|---|---|---|---|
| culture in | cells | % mast | cells | % mast |
| days | 10⁶/ml | cells | 10⁶/ml | cells |
| 5 | 0.7 | 0 | 3 | 0 |
| 12 | 0.5 | rare | 0.6 | 70 |
| 19 | 0.9 | 5 | 0.4 | 80 |
| 26 | 0.4 | 30 | 0.4 | 80 |
| 33 | 0.5 | 70 | 0.3 | 80 |
| 40 | 0.7 | 80 | 0.3 | 80 |
| 47 | 0.3 | 80 | 0.4 | 80 |
| 54 | 0.6 | 90 | 0.4 | 90 |
| 60 | 1.3 | 100 | 0.6 | 90 |
| 67 | 1.7 | 100 | 1.1 | 100 |
| 74 | 1 | 100 | 0.8 | 100 |
| 82 | 1.8 | 100 | 1.1 | 100 |
| 89 | 1 | 100 | 0.8 | 100 |
| 90 | 1 | 100 | 0.7 | 100 |
| 97 | — | — | 1.3 | 100 |
| 104 | — | — | 0.9 | 100 |

The kinetics for the cultures, whatever their tissue origin, reveal that, after a period of 60 days (bone marrow) and 67 days (fetal liver), the cell population undergoes a substantial increase, in particular for the cells of medullary origin. This increase corresponds to th change from a mixed culture to a 100% homogeneous population of mast cells.

These cells remain capable of multiplying after freezing/thawing.

To date, after 714 days of culturing, the mast cells are viable and continue to multiply.

### Characterization of The Cells

The mast cells are revealed by staining with toludin blue, and May Grünwald and Giemsa staining. The heparin and histamine in the granulations are revealed by staining with berberin sulfate and with alcian blue/safranin.

The mast cells in culture appear rounded with a high nucleus-cytoplasm ratio. The nucleus is rounded, most commonly central or off-center, non-segmented; there are many granulations in the cytoplasm, deep purple to red in color; the number and size of the granulations vary: they are small and are relatively few in the cells considered to be immature; they are large and there are many of them in the cells considered to be mature.

After staining the berberin sulfate, stained granulations in the cytoplasm of the mast cells, corresponding to the granules containing heparin, are observed under a fluorescent microscope. The amount of heparin appears to be variable from one cell to another; this variation may be related to the maturation stage of the cells.

After staining with alcian blue-safranin, the heparin grains are stained red with safranin and the histamine grains are stained blue with alcian blue. In some cells, strong labeling for heparin is observed, as in the case of the berberin staining.

The morphological characterization was completed by observation by electron microscopy.

The pig mast cells observed are cells which are large in size (16 μm). They have a large clear nucleus, which is often off-center and has a regular outline, with a chromatin slightly at the margins. One or two centrally positioned large nucleoles are observed in the nucleus. The cytoplasm is quite abundant; it contains ribosomes, many large mitochondria and many granulations.

The granulations appear to be surrounded by a single membrane containing a more or less electron-dense material, which is uneven in appearance, with small or medium-sized particles, sometimes structures in the form of more or less regular rods. These granulations are of the same type as those described in human mast cells in culture (EGUCHI, Electron. Microsc. Rev., 4(2), 293-318,1991).

Demonstration of the c-Kit Receptor and of the IgE Receptor

The presence of these receptors was demonstrated by immunohistochemistry.

c-Kit Receptor

This receptor was detected using the rabbit anti-human c-Kit polyclonal antibody C19 (TEBU), which also recognizes the porcine c-kit receptor.

Indirect staining at various culture times was carried out on slides after the cells had been cytospun. Two polyclonal antibodies were used: 1^{st}) the antibody C19, 2^{nd}) the FITC (fluorescene) goat-anti-rabbit antibody (TEBU). HMCI cells (originating from a human mastocytome) were used as positive control.
After cytospin, the cells are fixed with methanol for 5 min.
They are then air-dried and rinsed twice in PBS+1% BSA for 5 min.
They are then incubated with 5% goat serum for 20 min at ambient temperature in a humid chamber.
They are rinsed twice for 5 min. in PBS+1% BSA.
They are incubated with the 1^{st} antibody (C 19 at 1/50) for 1 h 30 min., at 37° C. in a humid chamber.
They are rinsed twice in PBS+l% BSA 2×5 min.
They are incubated with the 2^{nd} antibody (FITC goat anti-rabbit at 1/200) for 1 h 30 min., at 37° C. in a humid chamber.
They are rinsed twice for 5 min., in PBS.
The slides are mounted in mowiol.

They are read under a fluorescence microscope.

Positive membrane staining is observed on virtually all the cells; this staining is identical to that observed on the control HMCI cells, which shows that the cells analyzed possess c-kit receptors.

IgE Receptor

Direct staining at various culture times was carried out by flow cytometry on the mast cells in culture, with an FITC anti-mouse IgE antibody. RBL cells (line of rat basophils transformed into mast cells) were used as positive control.
1 ml of cells in culture, at approximately 10⁶ cells/ml, is used for each tube.
The cells are centrifuged to remove the culture medium.
The pellets are taken up in 1 ml of PBS+1% BSA, and incubated for 30 min., at +4° C.
The tubes are centrifuged and the pellets are taken up in 100 μl of FITC anti-mouse IgE antibody diluted to 4/100, and incubated for 1 h 30 min., at +4° C.
Rinsing is carried out in 2 ml of PBS, and the tubes are then centrifuged.
The pellets are collected in 1 ml of PBS.

The reading is carried out by FACS.

From D60 for the cultures derived from bone marrow, and from D67 for the cultures derived from liver, 99% of the cells are stained; the staining is identical on the control RBL cells. It therefore appears that the cells analyzed possess the IgE receptor.

At D639, the FACS profiles show, for the liver mast cells, two cell populations; a population which weakly expresses the IgE receptors, (17.5%) and a population which strongly expresses the IgE receptors, 82.5%.

Analysis of the Genomic DNA of the Cells

20 microsatellites from the diversified panel of the pig genome were analyzed using the DNA of the cells in culture (taken 489 days after the cells had been placed in culture) and using that of the boar and of the sow which were the parents of the 7 fetuses from which the liver and the bone marrow were taken. The markers chosen are very polymorphic and are distributed over virtually all the chromosomes.

The results obtained are as follows:
the Mendelian verification is satisfactory; the cells indeed originate from fetuses whose parents were identified;
for 4 main markers identified, IGFI, S0026, S0226 and SW240, the sizes observed correspond to the major alleles;
whatever the marker, the genotypes of the various samples of DNA from the fetal liver and fetal marrow mast cell cultures are identical.

The genomic DNA of the cells was not modified during the culturing (at least over 489 days, the date on which the DNA samples were prepared).

### Isolation of Pure Populations of Mast Cells From Fetal Marrow or Liver From Pigs of the Large White Breed

Samples were taken from 5 fetuses of a Large White sow on the 77^{th} day of gestation; 7 livers and 8 femurs were taken.

The cells were isolated and placed in culture, from liver or from marrow, as described above for the Meishan breed fetal cells.

Pure cultures of bone marrow mast cells or of liver mast cells were obtained after culturing for 112 days.

### EXAMPLE 2

### Obtaining Lines of Pig Mast Cells Transfected with an Oncogene Immortalization of the Cells in Culture

A sample of 2×10⁵ fetal liver mast cells at 202 days of culturing is transfected by lipofection using the vector pLM13, pre-linearized with the Sca1 restriction enzyme, combined with an artificial liposome, FuGENE6 (BOEHRINGER-MANNHEIM). The vector pLM13 comprises the sequences encoding a thermosensitive mutant of the T oncogene (Tts) of the SV40 vacuolizing simian virus, and those encoding the neo gene (resistance to geneticin). These sequences are under the control of a cytomegalovirus promoter.

The presence of the Tts oncogene in the genomic DNA of the transfected cells was observed by PCR. The expression of Tts was revealed by RT-PCR using a preparation of messenger RNA from the same cells.

### Growth of the Transfected Mast Cells

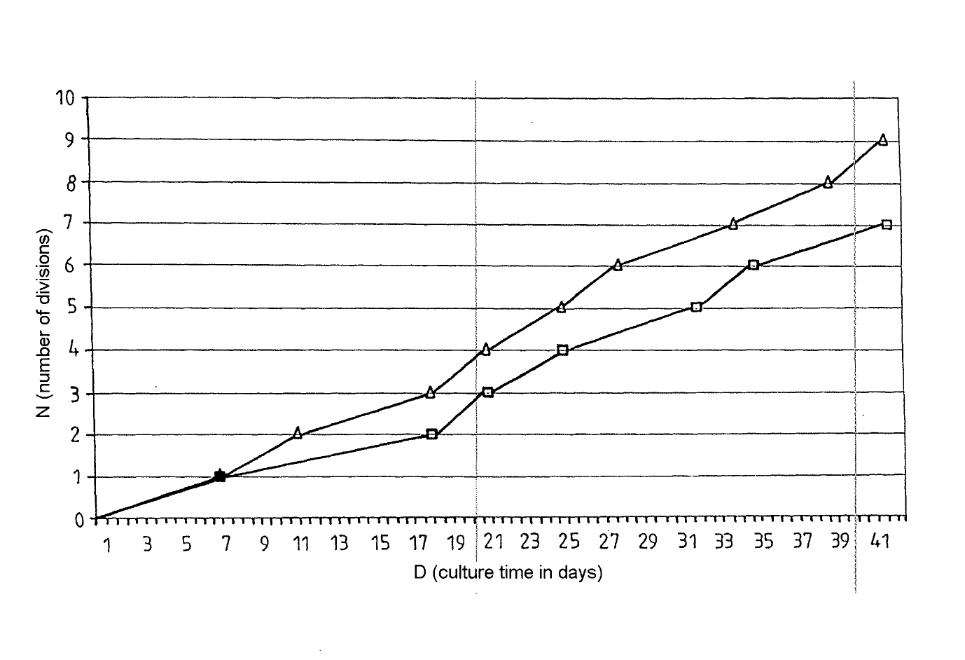

A growth curve was realized on the untransfected cells and on the transfected cells from D470 to D511, i.e. 41 days of culturing. Starting from an initial concentration of 5×10⁵ cells/ml, the cells were counted everyday and related to this initial concentration, in order to evaluate the number of divisions. The results are illustrated in FIG. 1. The culture time (in days) is given on the x-axis; the number of divisions is given on the y-axis.

A difference in growth is observed between the untransfected (□) and transfected (Δ) liver mast cells. The dividing time for the untransfected mast cells is on average 6 days, whereas it is 5 days for the transfected liver mast cells (FIG. 1).

### Characterization of the Lines Obtained

The cultures of transfected mast cells exhibit, in terms of their morphology and the presence of heparin, the same characteristics as the cultures of untransfected mast cells.

They also have the same c-kit receptor characteristics. However, as regards the IgE receptor, instead of the two cell populations observed at D639 in the case of the cultures of untransfected liver mast cells, a single population is observed.

### EXAMPLE 3

### Production of Histamine by the Mast Cells in Culture Assaying Histamine in the Mast Cells

Histamine was assayed in the three cell lines CNCM I-2735 (liver), CNCM I-2736 (liver transfected with the SV40 Tts oncogene) and CNCM I-2734 (bone marrow transfected with the SV40 Tts oncogene) at 976 days of culturing. The cells are placed in complete MEMa medium without serum.
1) Preparation of the cells:

50 000 cells of each line were activated for 30 minutes at 37° C. in the presence of 5% CO₂, with 500 μM of calcium ionophore A23187 and 5 μM of PMA (phorbol myristate acetate). The same number of cells of each line was placed at 37° C. in order to serve as a non-activated control.

The cells were then centrifuged and the supernatants and also the pellets were recovered and frozen at −80° C. until the time when the histamine was assayed.
2) Assaying the histamine:

The histamine was assayed in the supernatants and the pellets according to the technique described by LEBEL (Ann. Biochem. 133,16−29,1983).

The results are illustrated in table II below.

TABLE II

| | | HISTAMINE | |
|---|---|---|---|
| | | IN CELL | |
| | HISTAMINE IN | PELLET | HISTAMINE |
| LINE/ | SUPERNATANT | (ng/10⁶ | RELEASE |
| TREATMENT | (ng/ml) | cells) | (%) |
| I-2735 non-activated | 14 | 1583 | 48 |
| I-2735 activated | 449 | 466 | |
| I-2734 non-activated | 16 | 1460 | 28 |
| I-2734 activated | 250 | 600 | |
| I-2736 non-activated | 23 | 4380 | 25 |
| I-2736 activated | 593 | 1760 | |

All the cells contain histamine. The concentration of histamine in the supernatants is greater in the activated cells than in the non-activated cells.

The percentage histamine release by the activated cells is calculated using the following formula:

$$\% release = \frac{S - S Control \times 100}{( S + P ) - S Control}$$

in which: S=histamine in the supernatant P=histamine in the pellet

1: A method for obtaining a porcine mast cell culture, which comprises:
placing in culture stem cells obtained from pig fetal liver or bone marrow, in a medium comprising at least 0.2 ng/ml of porcine IL-3 and at least 8 ng/ml of porcine SCF.

2: The method as claimed in claim 1, wherein said cells are maintained in culture in said medium for at least 60 to 70 days.

3: The method as claimed in claim 1, which further comprises transforming cells of said culture with an immortalizing oncogene.

4: A porcine mast cell culture which can be obtained using a method as claimed in claim 1.

5: A porcine mast cell culture as claimed in claim 4, selected from the group consisting of the line deposited with the CNCM on Oct. 17, 2001, under the number I-2735; the line deposited with the CNCM on Oct. 17, 2001, under the number I-2736; the line deposited with the CNCM on Oct. 17, 2001, under the number I-2734.

6: The use of a porcine mast cell culture as claimed in claim 4, as a model for studying mast cell activation.

7: The use of a porcine mast cell culture as claimed in claim 4, for screening, in vitro, antigens capable of inducing mast cell activation.

8: A model for studying mast cell activation, which comprises:
the porcine mast cell culture as claimed in claim 4.

9: A model for studying mast cell activation, which comprises:
the porcine mast cell culture as claimed in claim 5.

10: An in vitro screening method, which comprises:
contacting the porcine mast cell culture as claimed in claim 4 with one or more antigen capable of inducing mast cell activation.

11: An in vitro screening method, which comprises:
contacting the porcine mast cell culture as claimed in claim 5 with one or more antigen capable of inducing mast cell activation.
